SEQUENCE LISTING (100 sequences)

SEQ ID NO: 1 - DNA, 60 bp, synthetic construct
  source 1..60: mol_type = other DNA
aattggacgtggctttcgcgttagtacgtagcatggtcacacaagcacagtagatcctgc

SEQ ID NO: 2 - DNA, 60 bp, synthetic construct
  source 1..60: mol_type = other DNA
tcataaggagtccggtgtagcgaaagatcaaggcgaccctaggtagcaaccgccggctcc

SEQ ID NO: 3 - DNA, 60 bp, synthetic construct
  source 1..60: mol_type = other DNA
cttccgctgggatccaacgttggcggccgaagccgccattccatagtgagtccttcgtct

SEQ ID NO: 4 - DNA, 60 bp, synthetic construct
  source 1..60: mol_type = other DNA
tcctagataccgcactctgggcagtacgaggtaatgccagtcacccagtgccgaacaaca

SEQ ID NO: 5 - DNA, 60 bp, synthetic construct
  source 1..60: mol_type = other DNA
tccagttcggtcagtgggtcactgcaagtagtcgattgcattgccaatctccgagtgatt

SEQ ID NO: 6 - DNA, 60 bp, synthetic construct
  source 1..60: mol_type = other DNA
aaaatcaccagtgcccaagaccaggggggctcgccgcgttggctaatcccggtacatctt

SEQ ID NO: 7 - DNA, 60 bp, synthetic construct
  source 1..60: mol_type = other DNA
cgagcggcgcagccgattaggaccatgtagaacatttgttacaagacttcttttaaacac

SEQ ID NO: 8 - DNA, 60 bp, synthetic construct
  source 1..60: mol_type = other DNA
gcttagttcaacctcgaatacctcgtatcattgtgcacctgccggtcaccagccaacgat

SEQ ID NO: 9 - DNA, 60 bp, synthetic construct
  source 1..60: mol_type = other DNA
atgacatgtggatgggcagtggccggttgttacacgcctgccgcgacgctgaatgacccg

SEQ ID NO: 10 - DNA, 60 bp, synthetic construct
  source 1..60: mol_type = other DNA
cggtctagctgactgtctatcgcctaggtcaaatagggagctttgatatctgcgtgtcca

SEQ ID NO: 11 - DNA, 60 bp, synthetic construct
  source 1..60: mol_type = other DNA
agtttatcccaccaaactatagccgtacaggccgaaatcttaagtcatatcgcgcgacta

SEQ ID NO: 12 - DNA, 60 bp, synthetic construct
  source 1..60: mol_type = other DNA
gcgtgcccagggtatattaggtcagcatcggatggactgacatgaacctttacaccgaag

SEQ ID NO: 13 - DNA, 60 bp, synthetic construct
  source 1..60: mol_type = other DNA
gtccacccgaccgtacatagaaatgagggtccccgtacgcccacgcacctgttcgctcgt

SEQ ID NO: 14 - DNA, 60 bp, synthetic construct
  source 1..60: mol_type = other DNA
gtcaccatgtaccaagggcgataacgatcggtgggagtattcatcgtggtgaagacgctg

SEQ ID NO: 15 - DNA, 60 bp, synthetic construct
  source 1..60: mol_type = other DNA
gccggcgtggaaggtaacagcaccgctgcgagcctaatgcgccgtttccacgaacacagg

SEQ ID NO: 16 - DNA, 60 bp, synthetic construct
  source 1..60: mol_type = other DNA
tcctggcctgcttgatgtctcgtgaccttcttagagatggacgaaatgtttcgcgaccta

SEQ ID NO: 17 - DNA, 60 bp, synthetic construct
  source 1..60: mol_type = other DNA
aggacctctagctcctttacaaagtgctggttccctttccggcgggatgccttatctaaa

SEQ ID NO: 18 - DNA, 60 bp, synthetic construct
  source 1..60: mol_type = other DNA
gtataatgctggagccctcccccaagcgttcagggtggggtttgctacgacttccgagtc

SEQ ID NO: 19 - DNA, 60 bp, synthetic construct
  source 1..60: mol_type = other DNA
aagtggcagcctaaacgatgttgggggctcgcgatgcacacgctctggtacaatacatac

SEQ ID NO: 20 - DNA, 60 bp, synthetic construct
  source 1..60: mol_type = other DNA
caaaagctttaaacgcgagttcccgcccataacctggaccgaatgcgggatcatgcatcg

SEQ ID NO: 21 - DNA, 40 bp, synthetic construct
  source 1..40: mol_type = other DNA
ccgcgcatcctatgtatcaagttagttctaatggaatacg

SEQ ID NO: 22 - DNA, 40 bp, synthetic construct
  source 1..40: mol_type = other DNA
ggcggtaaggtatcactcaagaagcaggctcggtaagaca

SEQ ID NO: 23 - DNA, 40 bp, synthetic construct
  source 1..40: mol_type = other DNA
gtgactatctgtgccagatcgtctggcagactgccgatcc

SEQ ID NO: 24 - DNA, 40 bp, synthetic construct
  source 1..40: mol_type = other DNA
cctgacctaacggtaagaggctcacataatggctccgccg

SEQ ID NO: 25 - DNA, 40 bp, synthetic construct
  source 1..40: mol_type = other DNA
tagcgtgacggccgcaggggacccataaaatgcgatcgta

SEQ ID NO: 26 - DNA, 40 bp, synthetic construct
  source 1..40: mol_type = other DNA
gtaatgaatattcagtagaaagtctgtgttagagggacga

SEQ ID NO: 27 - DNA, 40 bp, synthetic construct
  source 1..40: mol_type = other DNA
tatcttcctgcccagtggcggatggttatcgctgttgcta

SEQ ID NO: 28 - DNA, 40 bp, synthetic construct
  source 1..40: mol_type = other DNA
gtgcggacggcgttgcaacttccagggcctaatctgaccg

SEQ ID NO: 29 - DNA, 40 bp, synthetic construct
  source 1..40: mol_type = other DNA
gactagagtggcgagaactatggcgtgtgacccgttatgc

SEQ ID NO: 30 - DNA, 40 bp, synthetic construct
  source 1..40: mol_type = other DNA
gccttaggattcacttcagcgcgcaggcctgggtcgagat

SEQ ID NO: 31 - DNA, 40 bp, synthetic construct
  source 1..40: mol_type = other DNA
ggctcagctctatttttgtggtcatgggttctggtccgcc

SEQ ID NO: 32 - DNA, 40 bp, synthetic construct
  source 1..40: mol_type = other DNA
cggaaacgggtgcgtggactagcgaggagcaaacgaaaat

SEQ ID NO: 33 - DNA, 40 bp, synthetic construct
  source 1..40: mol_type = other DNA
cgtttgcatttaagggccgcacgaaccacagagcataagg

SEQ ID NO: 34 - DNA, 40 bp, synthetic construct
  source 1..40: mol_type = other DNA
ggtttacgtgggaaaggtgcttgtgtcccaacaggctagg

SEQ ID NO: 35 - DNA, 40 bp, synthetic construct
  source 1..40: mol_type = other DNA
gctgtccgatcgtatattaggactccgcgatggggttggc

SEQ ID NO: 36 - DNA, 40 bp, synthetic construct
  source 1..40: mol_type = other DNA
ggaagaggtcgccctacaaaatagatttgcgctactctcc

SEQ ID NO: 37 - DNA, 40 bp, synthetic construct
  source 1..40: mol_type = other DNA
cgcaatgacggacgtattcctctggccacatcggttcctg

SEQ ID NO: 38 - DNA, 40 bp, synthetic construct
  source 1..40: mol_type = other DNA
caacgtgtccgtgttcacgttatatgcgcacaaggccgag

SEQ ID NO: 39 - DNA, 40 bp, synthetic construct
  source 1..40: mol_type = other DNA
gtgttccggctgttatcctgcatcggaacgtcaatcatgc

SEQ ID NO: 40 - DNA, 40 bp, synthetic construct
  source 1..40: mol_type = other DNA
ttccactgtgtttgtctcatgtaggacgggcgcaaagcgt

SEQ ID NO: 41 - DNA, 25 bp, synthetic construct
  source 1..25: mol_type = other DNA
aattggacgtggctttcgcgttagt

SEQ ID NO: 42 - DNA, 25 bp, synthetic construct
  source 1..25: mol_type = other DNA
tcataaggagtccggtgtagcgaaa

SEQ ID NO: 43 - DNA, 25 bp, synthetic construct
  source 1..25: mol_type = other DNA
cttccgctgggatccaacgttggcg

SEQ ID NO: 44 - DNA, 25 bp, synthetic construct
  source 1..25: mol_type = other DNA
tcctagataccgcactctgggcagt

SEQ ID NO: 45 - DNA, 25 bp, synthetic construct
  source 1..25: mol_type = other DNA
tccagttcggtcagtgggtcactgc

SEQ ID NO: 46 - DNA, 25 bp, synthetic construct
  source 1..25: mol_type = other DNA
aaaatcaccagtgcccaagaccagg

SEQ ID NO: 47 - DNA, 25 bp, synthetic construct
  source 1..25: mol_type = other DNA
cgagcggcgcagccgattaggacca

SEQ ID NO: 48 - DNA, 25 bp, synthetic construct
  source 1..25: mol_type = other DNA
gcttagttcaacctcgaatacctcg

SEQ ID NO: 49 - DNA, 25 bp, synthetic construct
  source 1..25: mol_type = other DNA
atgacatgtggatgggcagtggccg

SEQ ID NO: 50 - DNA, 25 bp, synthetic construct
  source 1..25: mol_type = other DNA
cggtctagctgactgtctatcgcct

SEQ ID NO: 51 - DNA, 25 bp, synthetic construct
  source 1..25: mol_type = other DNA
agtttatcccaccaaactatagccg

SEQ ID NO: 52 - DNA, 25 bp, synthetic construct
  source 1..25: mol_type = other DNA
gcgtgcccagggtatattaggtcag

SEQ ID NO: 53 - DNA, 25 bp, synthetic construct
  source 1..25: mol_type = other DNA
gtccacccgaccgtacatagaaatg

SEQ ID NO: 54 - DNA, 25 bp, synthetic construct
  source 1..25: mol_type = other DNA
gtcaccatgtaccaagggcgataac

SEQ ID NO: 55 - DNA, 25 bp, synthetic construct
  source 1..25: mol_type = other DNA
gccggcgtggaaggtaacagcaccg

SEQ ID NO: 56 - DNA, 25 bp, synthetic construct
  source 1..25: mol_type = other DNA
tcctggcctgcttgatgtctcgtga

SEQ ID NO: 57 - DNA, 25 bp, synthetic construct
  source 1..25: mol_type = other DNA
aggacctctagctcctttacaaagt

SEQ ID NO: 58 - DNA, 25 bp, synthetic construct
  source 1..25: mol_type = other DNA
gtataatgctggagccctcccccaa

SEQ ID NO: 59 - DNA, 25 bp, synthetic construct
  source 1..25: mol_type = other DNA
aagtggcagcctaaacgatgttggg

SEQ ID NO: 60 - DNA, 25 bp, synthetic construct
  source 1..25: mol_type = other DNA
caaaagctttaaacgcgagttcccg

SEQ ID NO: 61 - DNA, 25 bp, synthetic construct
  source 1..25: mol_type = other DNA
cgtattccattagaactaacttgat

SEQ ID NO: 62 - DNA, 25 bp, synthetic construct
  source 1..25: mol_type = other DNA
tgtcttaccgagcctgcttcttgag

SEQ ID NO: 63 - DNA, 25 bp, synthetic construct
  source 1..25: mol_type = other DNA
ggatcggcagtctgccagacgatct

SEQ ID NO: 64 - DNA, 25 bp, synthetic construct
  source 1..25: mol_type = other DNA
cggcggagccattatgtgagcctct

SEQ ID NO: 65 - DNA, 25 bp, synthetic construct
  source 1..25: mol_type = other DNA
tacgatcgcattttatgggtcccct

SEQ ID NO: 66 - DNA, 25 bp, synthetic construct
  source 1..25: mol_type = other DNA
tcgtccctctaacacagactttcta

SEQ ID NO: 67 - DNA, 25 bp, synthetic construct
  source 1..25: mol_type = other DNA
tagcaacagcgataaccatccgcca

SEQ ID NO: 68 - DNA, 25 bp, synthetic construct
  source 1..25: mol_type = other DNA
cggtcagattaggccctggaagttg

SEQ ID NO: 69 - DNA, 24 bp, synthetic construct
  source 1..24: mol_type = other DNA
gcataacgggaggccctggtagtt

SEQ ID NO: 70 - DNA, 24 bp, synthetic construct
  source 1..24: mol_type = other DNA
atctcgaccaggccctggcgctga

SEQ ID NO: 71 - DNA, 24 bp, synthetic construct
  source 1..24: mol_type = other DNA
ggcggaccagaggccctggcacaa

SEQ ID NO: 72 - DNA, 24 bp, synthetic construct
  source 1..24: mol_type = other DNA
attttcgtttaggccctggagtcc

SEQ ID NO: 73 - DNA, 24 bp, synthetic construct
  source 1..24: mol_type = other DNA
ccttatgctcaggccctgggcggc

SEQ ID NO: 74 - DNA, 24 bp, synthetic construct
  source 1..24: mol_type = other DNA
cctagcctgtaggccctgggcacc

SEQ ID NO: 75 - DNA, 25 bp, synthetic construct
  source 1..25: mol_type = other DNA
gccaaccccaaggccctggcctaat

SEQ ID NO: 76 - DNA, 24 bp, synthetic construct
  source 1..24: mol_type = other DNA
ggagagtagcaggccctggtttgt

SEQ ID NO: 77 - DNA, 24 bp, synthetic construct
  source 1..24: mol_type = other DNA
caggaaccgaggccctggaggaat

SEQ ID NO: 78 - DNA, 24 bp, synthetic construct
  source 1..24: mol_type = other DNA
ctcggccttgaggccctggacgtg

SEQ ID NO: 79 - DNA, 24 bp, synthetic construct
  source 1..24: mol_type = other DNA
gcatgattgaaggccctggcagga

SEQ ID NO: 80 - DNA, 24 bp, synthetic construct
  source 1..24: mol_type = other DNA
acgctttgcgaggccctggatgag

SEQ ID NO: 81 - DNA, 20 bp, synthetic construct
  source 1..20: mol_type = other DNA
cgtattccattagaactaac

SEQ ID NO: 82 - DNA, 20 bp, synthetic construct
  source 1..20: mol_type = other DNA
tgtcttaccgagcctgcttc

SEQ ID NO: 83 - DNA, 20 bp, synthetic construct
  source 1..20: mol_type = other DNA
ggatcggcagtctgccagac

SEQ ID NO: 84 - DNA, 20 bp, synthetic construct
  source 1..20: mol_type = other DNA
cggcggagccattatgtgag

SEQ ID NO: 85 - DNA, 20 bp, synthetic construct
  source 1..20: mol_type = other DNA
tacgatcgcattttatgggt

SEQ ID NO: 86 - DNA, 19 bp, synthetic construct
  source 1..19: mol_type = other DNA
tcgtccctctaacacagac

SEQ ID NO: 87 - DNA, 20 bp, synthetic construct
  source 1..20: mol_type = other DNA
tagcaacagcgataaccatc

SEQ ID NO: 88 - DNA, 20 bp, synthetic construct
  source 1..20: mol_type = other DNA
cggtcagattaggccctgga

SEQ ID NO: 89 - DNA, 19 bp, synthetic construct
  source 1..19: mol_type = other DNA
gcataacgggaggccctgg

SEQ ID NO: 90 - DNA, 24 bp, synthetic construct
  source 1..24: mol_type = other DNA
atctcgaccaggccctggcgctga

SEQ ID NO: 91 - DNA, 19 bp, synthetic construct
  source 1..19: mol_type = other DNA
ggcggaccagaggccctgg

SEQ ID NO: 92 - DNA, 19 bp, synthetic construct
  source 1..19: mol_type = other DNA
attttcgtttaggccctgg

SEQ ID NO: 93 - DNA, 19 bp, synthetic construct
  source 1..19: mol_type = other DNA
ccttatgctcaggccctgg

SEQ ID NO: 94 - DNA, 19 bp, synthetic construct
  source 1..19: mol_type = other DNA
cctagcctgtaggccctgg

SEQ ID NO: 95 - DNA, 19 bp, synthetic construct
  source 1..19: mol_type = other DNA
gccaaccccaaggccctgg

SEQ ID NO: 96 - DNA, 18 bp, synthetic construct
  source 1..18: mol_type = other DNA
ggagagtagcaggccctg

SEQ ID NO: 97 - DNA, 19 bp, synthetic construct
  source 1..19: mol_type = other DNA
caggaaccgaggccctgga

SEQ ID NO: 98 - DNA, 19 bp, synthetic construct
  source 1..19: mol_type = other DNA
ctcggccttgaggccctgg

SEQ ID NO: 99 - DNA, 19 bp, synthetic construct
  source 1..19: mol_type = other DNA
gcatgattgaaggccctgg

SEQ ID NO: 100 - DNA, 19 bp, synthetic construct
  source 1..19: mol_type = other DNA
acgctttgcgaggccctgg

(19) United States
(12) Patent Application Publication
(10) Pub. No.: US 2026/0016479 A1
(43) Pub. Date: Jan. 15, 2026

(54) ELISA-PCR ASSAY WITH IMPROVED SENSITIVITY AND SPECIFICITY THROUGH SURFACE CAPTURE TECHNIQUE AND ITS APPLICATION

(71) Applicant: Suredx LLC, Carlsbad, CA (US)

(72) Inventors: Lei FANG, Carlsbad, CA (US); Lihao Meng, Carlsbad, CA (US); Allan Fang, San Diego, CA (US)

(21) Appl. No.: 18/915,618

(22) Filed: Oct. 15, 2024

Related U.S. Application Data
(60) Provisional application No. 63/670,154, filed on Jul. 12, 2024.

(51) Int. Cl. G01N 33/68; C12Q 1/6851; G01N 33/53

(52) U.S. Cl. CPC G01N 33/6803; C12Q 1/6851; G01N 33/5308

(57) ABSTRACT

An antibody-antigen capture ELISA-PCR test set for detection of a target protein comprises a first antibody-DNA conjugate having a first antibody which is configured to bind to a target protein; and a first DNA oligo linked to the first antibody; a second antibody-DNA conjugate having a second antibody which is configured to bind to the target protein; and a second DNA oligo linked to the second antibody; and multiple immobilized DNA fragments attached to a designated surface on their attaching ends, wherein the immobilized DNA fragments are complementary to one of the first DNA oligo and the second DNA oligo.

### CROSS-REFERENCE TO RELATED PATENT APPLICATIONS

This application claims priority to and benefit of U.S. Provisional Application Ser. No. 63/670,154 which is filed on Jul. 12, 2024, and hereby incorporated by reference in its entirety.

### SEQUENCE LISTING

A Sequence Listing conforming to the rules of WIPO Standard ST.26 is hereby incorporated by reference. Said Sequence Listing has been filed as an electronic document via PatentCenter encoded as XML in UTF-8 text. The electronic document, created on Oct. 7, 2024, is entitled “1009984117US2_NPA_SL.xml”, and is 90,210 bytes in size.

### FIELD OF THE INVENTION

The present invention relates to the field of molecular assays. More particular, the present invention relates to ELISA-PCR assay with improved sensitivity and specificity through surface antibody-antigen capture technique via an ELISA-PCR test platform.

### BACKGROUND OF THE INVENTION

The background description provided herein is for the purpose of presenting the context of the invention. The subject matter discussed in the background of the invention section should not be assumed to be prior art merely because of its mention in the background of the invention section. Similarly, a problem mentioned in the background of the invention section or associated with the subject matter of the background of the invention section should not be assumed to have been previously recognized in the prior art. The subject matter in the background of the invention section merely represents different approaches, which in and of themselves may also be inventions. Work of the presently named inventors, to the extent it is described in the background of the invention section, as well as aspects of the description that may not otherwise qualify as prior art at the time of filing, are neither expressly nor impliedly admitted as prior art against the invention.

Traditional immunoassay and molecular diagnosis are two major testing methods for detection of a target molecule (e.g. a target protein, DNA or RNA) in the field of medical diagnosis. Immunoassay is a selective bioanalytical method that measures the presence or concentration of a target protein (e.g. an enzymatic protein in a subject, or a capsid protein of a virus) through the use of an antibody or an antigen as a biorecognition agent, while, the molecular diagnosis, e.g. PCR test, has high sensitivity, high specificity, and its capability for quantitative analysis. Despite the rise of molecular diagnosis, immunoassays remain irreplaceable due to its unique advantages in protein-based detection, as well as its simple operation and low cost, as compared to the molecular diagnosis. However, both traditional immunoassay and molecular diagnosis are limited in its detection sensitivity and specificity, as well as its time consuming.

Previously we have disclosed a method of molecular assays using antibody and DNA oligo conjugates in detection of a target protein via an ELISA-PCR test platform, which has great potentials to be widely used in clinical and research settings to detect and quantify specific antigens. However, the sensitivity and specificity of the aforementioned ELISA-PCR test are still limiting their effectiveness.

Therefore, heretofore unaddressed needs exist in the art to address the aforementioned deficiencies and inadequacies.

### SUMMARY OF THE INVENTION

In light of the foregoing, this invention discloses an ELISA-PCR test, which combines the high specificity of the enzyme-linked reaction and the amplification of PCR in a breakthrough, high-sensitivity protein quantitative analysis.

In one aspect of the invention, an antibody-antigen capture ELISA-PCR test set for detection of a target protein comprises a first antibody-DNA conjugate having a first antibody which is configured to bind to a target protein; and a first DNA oligo linked to the first antibody; a second antibody-DNA conjugate having a second antibody which is configured to bind to the target protein; and a second DNA oligo linked to the second antibody; and multiple immobilized DNA fragments attached to a designated surface on their attaching ends, wherein the immobilized DNA fragments are complementary to one of the first DNA oligo and the second DNA oligo.

In one embodiment, the first antibody binds to a first binding site of the target protein and the second antibody binds to a second binding site of the target protein.

In one embodiment, the attaching ends of the immobilized DNA fragments are 5′ ends.

In one embodiment, the immobilized DNA fragments are configured to capture a complex formed by the first antibody-DNA conjugate, the second antibody-DNA conjugate, and the target protein.

In one embodiment, a blocking nucleotide is attached to each of the immobilized DNA fragments on an end opposite to its attaching end.

In one embodiment, each of the immobilized DNA fragments comprises a cleave site adjacent to the attaching end.

In one embodiment, each of the immobilized DNA fragment has a length between 15-30 nucleotide bases.

In one embodiment, each of the immobilized DNA fragment is attached to the designated surface via click chemistry or avidin-biotin interactions.

In one embodiment, the first DNA oligo is covalently linked to the first antibody, and the second DNA oligo is covalently linked to the second antibody.

In one embodiment, the first DNA oligo is covalently linked to the first antibody on its 3′ end, and the second DNA oligo is covalently linked to the second antibody on its 5′ end.

In one embodiment, the first DNA oligo and second DNA oligo each has a length between 20-300 nucleotide bases.

In one embodiment, when the first antibody and second antibody each binds to the target protein, the 5′ end of the first DNA oligo and the 3′ end of the second DNA oligo attaches to each other.

In one embodiment, the first antibody and second antibody, the first DNA oligo and second DNA oligo, and the target protein form a close loop structure.

In one embodiment, the target protein is selected from the group consisting of marker proteins of pathogens, marker proteins of infectious diseases; marker proteins of genetic diseases, marker proteins of acquired diseases.

In another aspect of the invention, a method for quantitatively detecting a target protein using antibody-antigen capture ELISA-PCR test comprises mixing a first antibody-DNA conjugate, a second antibody to a sample having the target protein to form a complex mixture; incubating the complex mixture on a designated surface on which immobilized DNA fragments are attached; washing the incubated designated surface and collected a concentrated complex mixture; and conduct an ELISA-PCR test using the concentrated complex mixture.

In one embodiment, the first antibody-DNA conjugate having a first antibody which is configured to bind to a first biding site of the target protein and a first DNA oligo linked to the first antibody; and the second antibody-DNA conjugate having a second antibody which is configured to bind to a second biding site of the target protein; and a second DNA oligo linked to the second antibody.

In one embodiment, when the first antibody and second antibody each binds to the target protein, a 3′ end of the first DNA oligo and a 5′ end of the second DNA oligo attach to each other.

In one embodiment, the ELISA-PCR test comprises adding a first primer and a second primer to the sample, wherein the first primer is complementary to a sequence at the 3′ end of the first DNA oligo, and the second primer is identical to a sequence at the 5′ end of the second DNA oligo.

In one embodiment, the ELISA-PCR test further comprises starting PCR cycle to amplify the first and second DNA oligos with the first and second primers.

In one embodiment, the ELISA-PCR test further comprises quantitatively analyzing the result of the PCR amplification to determine the amount of the target protein in the sample.

These and other aspects of the present invention will become apparent from the following description of the preferred embodiment taken in conjunction with the following drawings, although variations and modifications therein may be affected without departing from the spirit and scope of the novel concepts of the disclosure.

### BRIEF DESCRIPTION OF THE DRAWINGS

The accompanying drawings illustrate one or more embodiments of the invention and together with the written description, serve to explain the principles of the invention. Wherever possible, the same reference numbers are used throughout the drawings to refer to the same or like elements of an embodiment.

FIG. 1 provides a systematic illustration of the ELISA-PCR test using antibody-antigen capture technique.

FIG. 2 illustrates a chart showing the ELISA-PCR test results of ST2 (ST2).

FIG. 3 illustrates a chart showing the ELISA-PCR test result of Tau181.

### DETAILED DESCRIPTION OF THE INVENTION

The invention will now be described more fully hereinafter with reference to the accompanying drawings, in which exemplary embodiments of the invention are shown. This invention may, however, be embodied in many different forms and should not be construed as limited to the embodiments set forth herein. Rather, these embodiments are provided so that this invention will be thorough and complete, and will fully convey the scope of the invention to those skilled in the art. Like reference numerals refer to like elements throughout.

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated if the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the invention has been specifically disclosed by preferred embodiments and optional features, modification and variation of the concepts herein disclosed may be resorted to by those skilled in the art, and that such modifications and variations are considered to be within the scope of this invention as defined by the appended claims.

Whenever a range is given in the specification, for example, a temperature range, a time range, or a composition or concentration range, all intermediate ranges and subranges, as well as all individual values included in the ranges given are intended to be included in the invention. It will be understood that any subranges or individual values in a range or subrange that are included in the description herein can be excluded from the claims herein.

It will be understood that, as used in the description herein and throughout the claims that follow, the meaning of “a”, “an”, and “the” includes plural reference unless the context clearly dictates otherwise. Thus, for example, reference to “a cell” includes a plurality of such cells and equivalents thereof known to those skilled in the art. As well, the terms “a” (or “an”), “one or more” and “at least one” can be used interchangeably herein. It is also to be noted that the terms “comprising”, “including”, and “having” can be used interchangeably.

It will be understood that when an element is referred to as being “on”, “attached” to, “connected” to, “coupled” with, “contacting”, etc., another element, it can be directly on, attached to, connected to, coupled with or contacting the other element or intervening elements may also be present. In contrast, when an element is referred to as being, for example, “directly on”, “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of the invention.

It will be further understood that the terms “comprises” and/or “comprising”, or “includes” and/or “including”, or “has” and/or “having”, or “carry” and/or “carrying”, or “contain” and/or “containing”, or “involve” and/or “involving”, “characterized by”, and the like are to be open-ended, i.e., to mean including but not limited to. When used in this disclosure, they specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the invention, and will not be interpreted in an idealized or overly formal sense unless expressly so defined herein.

As used in the disclosure, “around”, “about”, “approximately” or “substantially” shall generally mean within 20 percent, preferably within 10 percent, and more preferably within 5 percent of a given value or range. Numerical quantities given herein are approximate, meaning that the terms “around”, “about”, “approximately” or “substantially” can be inferred if not expressly stated.

As used in the disclosure, the phrase “at least one of A, B, and C” should be construed to mean a logical (A or B or C), using a non-exclusive logical OR. As used herein, the term “and/or” includes any and all combinations of one or more of the associated listed items.

As used in the disclosure, the term “amino acid” is intended to mean both naturally occurring and non-naturally occurring amino acids as well as amino acid analogs and mimetics. Naturally occurring amino acids include the 20 (L)-amino acids utilized during protein biosynthesis as well as others such as 4-hydroxyproline, hydroxylysine, desmosine, isodesmosine, homocysteine, citrulline and ornithine, for example. Non-naturally occurring amino acids include, for example, (D)-amino acids, norleucine, norvaline, p-fluorophenylalanine, ethionine and the like, which are known to a person skilled in the art. Amino acid analogs include modified forms of naturally and non-naturally occurring amino acids. Such modifications can include, for example, substitution or replacement of chemical groups and moieties on the amino acid or by derivatization of the amino acid. Amino acid mimetics include, for example, organic structures which exhibit functionally similar properties such as charge and charge spacing characteristic of the reference amino acid. For example, an organic structure which mimics Arginine (Arg or R) would have a positive charge moiety located in similar molecular space and having the same degree of mobility as thee-amino group of the side chain of the naturally occurring Arg amino acid. Mimetics also include constrained structures so as to maintain optimal spacing and charge interactions of the amino acid or of the amino acid functional groups. Those skilled in the art know or can determine what structures constitute functionally equivalent amino acid analogs and amino acid mimetics.

As used in the disclosure, the terms “polypeptide” and “protein” are used interchangeably herein to refer to a polymer of amino acid residues and to variants and synthetic analogues of the same. Thus, these terms apply to amino acid polymers in which one or more amino acid residues are synthetic non-naturally occurring amino acids, such as a chemical analogue of a corresponding naturally occurring amino acid, as well as to naturally-occurring amino acid polymers. The polypeptides described herein are not limited to a specific length of the product; thus, peptides, oligopeptides, and proteins are included within the definition of polypeptide, and such terms may be used interchangeably herein unless specifically indicated otherwise. The polypeptides described herein may also comprise post-expression modifications, such as glycosylations, acetylations, phosphorylations and the like, as well as other modifications known in the art, both naturally occurring and non-naturally occurring. A polypeptide may be an entire protein, or a subsequence, fragment, variant, or derivative thereof.

As used in the disclosure, the term “oligonucleotides (oligonucleotide)” or “oligonucleotides (oligo)” refer to short nucleotide sequences.

As used in the disclosure, nucleic acid (for example, DNA) molecule is referred to as having “5′ end” and “3′ end”, because mononucleotide is with such side Formula is reacted to produce oligonucleotides or polynucleotides: described mode makes 5′ phosphoric acid of a mononucleotide pentose ring with a side To the 3′ oxygen being attached to its ortho position mononucleotide pentose ring via phosphodiester bond. Therefore, the end of oligonucleotides or polynucleotides End, if its 5′ phosphoric acid is not connected with 3′ oxygen of mononucleotide pentose ring, be then referred to as “5′ end”, and if its 3′ oxygen not with 5′ phosphoric acid of mononucleotide pentose ring subsequently connect, then be referred to as “3′ end”. As used herein, nucleotide sequence, even if inside at bigger oligonucleotides or polynucleotides, it is possible to be referred to as that there are 5′ and 3′ ends. At linear or ring-shaped DNA molecule In, the discrete element is referred to as “upstream” or the 5′ ends of “downstream” or 3′ end element. This term reflects transcribes with 5′ ends to 3′ end mode along DNA carrying out the fact. The promoter transcribed of the gene connecting and enhancer element is instructed to be usually located at 5′ ends of the code area or upstream. But enhancer element even also can be sent out when being positioned at the 3′ end of the promoter element and code area Wave its effect. Transcription termination and polyadenylation signals are positioned at 3′ ends or the downstream of the coding area.

As used in the disclosure, the term “conjugate” is intended to refer to the entity formed as a result of covalent or non-covalent attachment or linkage of an antibody or other molecule, e.g., a DNA oligo attached to an antibody. One example of a conjugate is an “antibody-DNA conjugates”, that is, an antibody covalently linked to at least one DNA oligo of 20-300 bases.

As used in the disclosure, the terms “function” and “functional” and the like refer to a biological, enzymatic, or therapeutic function.

As used in the disclosure, “homology” refers to the percentage number of amino acids that are identical or constitute conservative substitutions. Homology may be determined using sequence comparison programs such as GAP (Deveraux et al., Nucleic Acids Research. 12, 387-395, 1984), which is incorporated herein by reference. In this way sequences of a similar or substantially different length to those cited herein could be compared by insertion of gaps into the alignment, such gaps being determined, for example, by the comparison algorithm used by GAP.

As used in the disclosure, “isolated” refers to material that is substantially or essentially free from components that normally accompany it in its native state. For example, an “isolated peptide” or an “isolated polypeptide”, “isolated DNA” and the like, as used herein, includes the in vitro isolation and/or purification of a peptide, polypeptide, DNA molecule from its natural cellular environment, and from association with other components of the cell; i.e., it is not significantly associated with in vivo substances.

The term “linkage,” “linker,” “linker moiety,” or “L” is used herein to refer to a linker that can be used to separate an antibody or a fragment of the antibody from DNA, or to separate a first agent from another agent, for instance where two or more agents are linked to form an antibody-DNA conjugate. The linker may be physiologically stable or may include a releasable linker such as an enzymatically degradable linker (e.g., proteolytically cleavable linkers). In certain aspects, the linker may be a peptide linker, for instance, as part of an antibody protein. In some aspects, the linker may be a non-peptide linker or non-proteinaceous linker, e.g. a DNA. In some aspects, the linker may be particle, such as a nanoparticle.

As used in the disclosure, the terms “modulating” and “altering” include “increasing,” “enhancing” or “stimulating,” as well as “decreasing” or “reducing,” typically in a statistically significant or a physiologically significant amount or degree relative to a control. An “increased,” “stimulated” or “enhanced” amount is typically a “statistically significant” amount, and may include an increase that is 1.1, 1.2, 2, 3, 4, 5, 6, 7, 8, 9, 10, 15, 20, 30 or more times (e.g., 500, 1000 times) (including all integers and decimal points in between and above 1, e.g., 1.5, 1.6, 1.7.1.8, etc.) the amount produced by no composition (e.g., the absence of polypeptide of conjugate of the invention) or a control composition, sample or test subject. A “decreased” or “reduced” amount is typically a “statistically significant” amount, and may include a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% decrease in the amount produced by no composition or a control composition, including all integers in between.

As used in the disclosure, in certain embodiments, the “purity” of any given agent in a composition may be specifically defined. For instance, certain compositions may comprise an agent that is at least 80%, 85%, 90%, 91%, 92%, 93%, 94%, 95%, 96%, 97%, 98%, 99%, or 100% pure, including all decimals in between, as measured, for example and by no means limiting, by high pressure liquid chromatography (HPLC), a well-known form of column chromatography used frequently in biochemistry and analytical chemistry to separate, identify, and quantify compounds.

As used in the disclosure, the terms “sample” and “samples” are used with its broadest sense herein and can include biological sample and ring Border sample. Patient Sample A can include the sample of all types obtained from mankind and other animals, and including but not limited to, body fluid is all such as urine, blood (whole blood, serum and plasma), fecal matter, cerebrospinal fluid (CSF), seminal fluid and saliva and solid tissue. These biological samples can also be material originate from patients but further developed in vitro, such as cell culture and tissue culture. Biological samples can be animal, including the mankind, fluid or tissue.

As used in the disclosure, the term “thermal cycler” refers to a programmable thermal cycler instrument, such as used for performing PCR Device.

As used in the disclosure, the term “amplifying reagent” can refer to those reagents (such as DNA required for amplification of nucleic acid sequences Polymerase, deoxyribonucleoside triphosphate, buffer solution etc.). A “physiologically cleavable” or “hydrolyzable” or “degradable” bond is a bond that reacts with water (i.e., is hydrolyzed) under physiological conditions. The tendency of a bond to hydrolyze in water will depend not only on the general type of linkage connecting two central atoms but also on the substituents attached to these central atoms. Appropriate hydrolytically unstable or weak linkages include, but are not limited to: carboxylate ester, phosphate ester, anhydride, acetal, ketal, acyloxy alkyl ether, imine, orthoester, thioester, thiol ester, carbonate, and hydrazone, peptides and oligonucleotides.

As used in the disclosure, the term “reference sequence” refers generally to a nucleic acid coding sequence, or amino acid sequence, to which another sequence is being compared. All polypeptide and polynucleotide sequences described herein are included as references sequences, including those described by name and those described in the Tables and the Sequence Listing.

As used in the disclosure, the terms “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, lie, Phe, Tyr, Trp, Lys, Arg, His, Asp, Glu, Asn, Gin, Cys and Met) occurs in both sequences to yield the number of matched positions, dividing the number of matched positions by the total number of positions in the window of comparison (i.e., the window size), and multiplying the result by 100 to yield the percentage of sequence identity. Included are nucleotides and polypeptides having at least about 50%, 55%, 60%, 65%, 70%, 75%, 80%, 85%, 90%, 95%, 97%, 98%, 99% or 100% sequence identity to any of the reference sequences described herein (see, e.g., Sequence Listing), typically where the polypeptide variant maintains at least one biological activity of the reference polypeptide.

As used in the disclosure, terms used to describe sequence relationships between two or more polynucleotides or polypeptides include “reference sequence,” “comparison window,” “sequence identity,” “percentage of sequence identity,” and “substantial identity.” A “reference sequence” is at least 12 but frequently 15 to 18 and often at least 25 monomer units, inclusive of nucleotides and amino acid residues, in length.

As used in the disclosure, by “statistically significant,” it is meant that the result was unlikely to have occurred by chance. Statistical significance can be determined by any method known in the art. Commonly used measures of significance include the p-value, which is the frequency or probability with which the observed event would occur, if the null hypothesisis true. If the obtained p-value is smaller than the significance level, then the null hypothesis is rejected. In simple cases, the significance level is defined at a p-value of 0.05 or less.

As used in the disclosure, the term “complementary” is used for mentioning by base pairing rules related to manynucleotides (that is, nucleotide sequence). For example, for sequence “A-G-T”, complementary with sequence “T-C-A”. Complementarity can be “portion point”, some in the base of its amplifying nucleic acid are mated according to base pairing rules. Or, can exist between nucleic acids “completely (complete)” or “(total) just thoroughly” complementary.

As used in the disclosure, a “subject,” as used herein, includes any animal that exhibits a symptom, or is at risk for exhibiting a symptom, which can be diagnosed with an antibody-DNA oligo conjugate of the invention. Suitable subjects (patients) include laboratory animals (such as mouse, rat, rabbit, or guinea pig), farm animals, and domestic animals or pets (such as a cat or dog). Non-human primates and, preferably, human patients, are included.

As used in the disclosure, “substantially” or “essentially” means nearly totally or completely, for instance, 95%, 96%, 97%, 98%, 99% or greater of some given quantity.

As used in the disclosure, “substantially free” refers to the nearly complete or complete absence of a given quantity for instance, less than about 10%, 5%, 4%, 3%, 2%, 1%, 0.5% or less of some given quantity. For example, certain compositions may be “substantially free” of cell proteins, membranes, nucleic acids, endotoxins, or other contaminants.

As used in the disclosure, the term “wild-type” refers to a gene or gene product that has the characteristics of that gene or gene product when isolated from a naturally-occurring source. A wild type gene or gene product (e.g., a polypeptide) is that which is most frequently observed in a population and is thus arbitrarily designed the “normal” or “wild-type” form of the gene.

As used in the disclosure, the terms “real-time” and “real-time continuous” are interchangeable and refer to a method in which data collection occurs during the course of a polymerization reaction by periodic monitoring. Thus, the method combines amplification and detection into a single step.

As used in the disclosure, the term “quantitative PCR” or “qPCR” refers to the use of the Polymerase Chain Reaction (PCR) to quantify target DNA.

As used in the disclosure, the term “Ct” and “cycle threshold” refer to the time at which the fluorescence intensity is greater than background fluorescence. Characterized by the time point (or PCR cycle) at which the amplification of the target is first detected. Thus, the greater the amount of target DNA in the starting material, the faster a significant increase in fluorescence signal will occur, resulting in a lower Ct.

Embodiments of the invention are illustrated in detail hereinafter with reference to accompanying drawings. The description below is merely illustrative in nature and is in no way intended to limit the invention, its application, or uses. The broad teachings of the invention can be implemented in a variety of forms. Therefore, while this invention includes particular examples, the true scope of the invention should not be so limited since other modifications will become apparent upon a study of the drawings, the specification, and the following claims. For purposes of clarity, the same reference numbers will be used in the drawings to identify similar elements. It should be understood that one or more steps within a method may be executed in different order (or concurrently) without altering the principles of the invention.

The current invention introduces an ELISA-PCR test integrated with an antibody-antigen capture system to increase assay sensitivity and specificity.

In certain embodiments, the present invention significantly improves the performance of ELISA-PCR assays by capturing antibody-antigen complexes on a designated surface such as magnetic bead surfaces or walls of qPCR plate wells. This technique utilizes DNA fragments that are reverse-complementary to DNA oligonucleotides of antibody-DNA conjugates to capture any complexes formed by the antibody-DNA conjugates whose DNA oligonucleotides are captured by the DNA fragments immobilized on the designated surface. This method not only selectively enriches target complexes but also minimizes background, and therefore augmenting assay sensitivity by 10 to 100 times.

### Antibody-DNA Conjugates

In certain embodiments, the invention provides a methodology to covalently label antibodies with DNA molecules of 20-300 bases in a site-specific way for downstream applications, such as Immuno-PCR, Proximity Ligation PCR or any immuno-assays using antibody-DNA conjugates as probes.

In one embodiment of the invention, an antibody-DNA conjugate comprises one or more DNA molecules which are covalently attached to a heavy chain of an antibody. Each of these antibody-DNA conjugates has 1 to 10 DNA molecules per antibody. This labeling method does not interfere with the epitope binding site of the antibody so as to ensure the antibody binding affinity and specificity. This labeling method generates covalently labeled antibodies used in ELISA-PCR test. Comparing to traditional ELISA test, the ELISA-PCR test using the antibody-DNA conjugates increases the detection sensitivity of a target protein by 10 times or more.

In one embodiment of the invention, the labeling method for the antibody-DNA conjugates used in the ELISA-PCR test includes the following steps:

(1) Modify a DNA oligo with one or more chemicals to form a DNA oligo with an active functional group. In one embodiment of the invention, during this reaction, the DNA oligo molecule is attached with an —NH₂ functional group at the 3′ or the 5′ end. The DNA-NH₂ is then incubated with DBCO, TCO, Tetrazines, Azide, Alkynes, or similar chemicals for 1-48 hours at room temperature (RT) around 25° C. at a ratio of 1:10. In some embodiments, the ratio of DNA-NH₂ to the DBCO, TCO, Tetrazines, Azide, Alkynes, or similar chemicals ranges between 1:2 to 1:20.

(2) Modify antibody using click chemistry by adding 1-10 azide groups to the heavy chains of the antibody molecule. This process involves modifying the antibody heavy chain through an enzymatic step. For example, 1-10 microgram of Gal-T transferase was used per reaction with 100-10,000 microgram UDG-GalNaz to incubate with 100 to 1000 microgram antibody at approximate 30° C. overnight (>=18 hours).

(3) Incubate the DNA oligo(s) obtained from step (1) with the activated antibody obtained from step (2) to form the final product: a site-specific, covalently labeled antibody with 1-10 DNA oligo(s). In one embodiment, the 3′ or the 5′ end of the DNA oligo(s) with the —NH₂ functional group is/are attached to the carbohydrate group(s) of the antibody which is activated.

In one example of the invention, the antibody may be a Tau antibody, e.g., Tau181 (PA5114656, Thermo Fisher), which specifically binds to protein Tau and is used as a marker of Alzheimer's disease. In one example of the invention, one or more DNA oligos are covalently linked to the antibody Tau181 antibody.

It should be noted that the Tau181 antibody is only used as an example. Other antibodies, either commercially available or lab-generated, can be labeled via functional groups and thereafter covalently linked with DNA oligos for being used in the ELISA-PCR test to detect different target proteins.

In certain embodiments, the antibody is selected from one or more of Tau antibodies on the market such as of e.g. Tau13 sc-21796 specific antibody, and Tau monoclonal antibody BT2 (Catalog #MN1010, Thermo Fisher), and etc.

In one embodiment, the antibody may be an antibody of a cytokine, e.g. ST2.

In other embodiments, antibodies binding to virus, bacteria, diseases markers can be used for the ELISA-PCR test.

Designated Surfaces Treated with DNA Fragments

To improve the sensitivity and specificity of ELISA-PCR test, the present invention utilizes designated surfaces on which DNA fragments partially or fully complementary to the DNA oligos of the antibody-DNA conjugates are immobilized. In one embodiment, these immobilized DNA fragments have about 15-30 nucleotide bases which are specially synthesized to be reverse-complementary to one or more DNA oligos of the antibody-DNA conjugates. In another embodiment, each of these immobilized DNA fragments has about 10-25 nucleotide bases. In another embodiment, each of these immobilized DNA fragments has about 20-35 nucleotide bases.

These immobilized DNA fragments selectively and efficiently capture one or more of the DNA oligos of the antibody-DNA conjugates together with any complex that the antibody-DNA conjugates may form.

In certain embodiments, the designated surfaces may be surfaces of synthesized magnetic beads or modify surfaces of qPCR plate wells. In certain embodiments, the designated surfaces may be surfaces of beads of other materials. In certain embodiments, each of the beads has a diameter between about 0.1 to 100 micrometer. In certain embodiments, each of the beads has a diameter between about 50 nanometer to 500 micrometer. In certain embodiments, the designated surfaces may be surfaces of commonly used objected in molecular analysis.

In certain embodiments, the immobilized DNA fragments are immobilized on to the designed surfaces using covalent bonding methods. In certain embodiments, the covalent bonding method includes click chemistry reaction involving reactions giving high yield and selectivity products by carbon-hetero bond formation reactions. In certain embodiments, the covalent bonding method includes avidin-biotin interactions. In certain embodiments, the covalent bonding method includes other commonly known methods for attaching DNA fragments to a solid or a semi-solid surface.

Examples of immobilized DNA fragments are disclosed in Table 3.

### Antibody-Antigen Capture ELISA-PCR Test

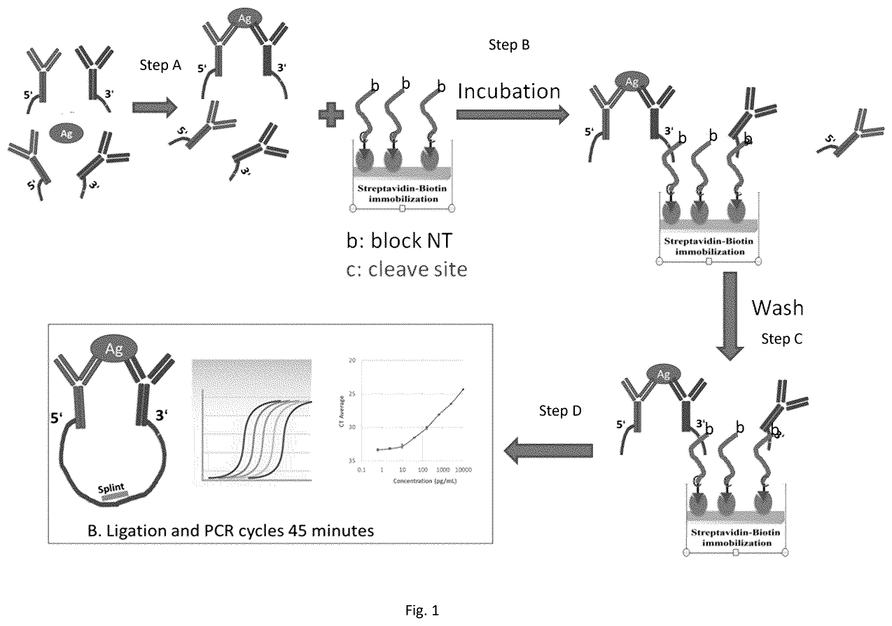

As illustrated in FIG. 1, in certain embodiments, an antibody-Antigen Capture ELISA-PCR test has stage (A), stage (B), stage (C) and stage (D).

In certain embodiments, for detection of a target protein using an antibody-antigen capture ELISA-PCR test, two different antibody-DNA conjugates are used.

In one embodiment of the invention, the 5′ end of a first DNA oligo is covalently linked to a first antibody and the 3′ end of a second DNA oligo is covalently linked to a second antibody. During stage (A), while the first antibody-DNA conjugate binds to a first specific region of the target protein, the second antibody-DNA conjugate binds to a second region of the target protein.

It should be noted that the first and second antibodies may bind to two separate binding sites of the target protein. Regardless of the binding site, the first and the second DNA oligos should have a length enough for the 3′ end of the first DNA oligo and the 5′ end of the second DNA oligo to be ligated to each other, when both of the first and the second antibodies bind to the target protein.

During stage (A), the first and the second antibody-DNA conjugates are mixed with a sample containing at least one target protein for detection. In certain embodiments, the sample contains more than one target proteins. During this stage, the antibodies of the first and the second antibody-DNA conjugates bind to the target protein in the sample, and form antigen-antibody-DNA complexes in the mixture. The mixture may also include any first and second antibody-DNA conjugates which have not bound to the target protein.

When there are more than one target proteins to be detected in the sample, one than one pair of the antibody-DNA conjugates are necessary. In certain embodiments, a first and a second antibody-DNA conjugate bind to a first target proteins, and a third and a fourth antibody-DNA conjugate bind to a second target proteins. The DNA oligos of the first and second antibody-DNA conjugates should be different from the DNA oligos of the third and fourth antibody-DNA conjugates such that the each pair of the forward and reverse primers used in the PCR tests later are capable of specifically binds to either the DNA oligos of the first and second antibody-DNA conjugates, or the DNA oligos of the third and fourth antibody-DNA conjugates, but never the both.

In certain embodiments, the mixture obtained above is incubated at a temperature between 0-40 Celsius degree for a period of between 2-120 minutes. In certain embodiments, the mixture obtained above is incubated at a temperature between 15-40 Celsius degree for a period of between 10-30 minutes.

During the stage (B), the mixture is contacted and incubated with a designated surface on which immobilized DNA fragments are attached. The incubation on the contacted designated surface is conducted at a temperature between 0-40 Celsius degree for a period of between 2-120 minutes for the immobilized DNA fragments to capture the complexes containing the DNA oligos to which they are complimentary.

Once the incubation is completed, one or more washing steps are conducted during the stage (C) to purge unbound components such as antibody-DNA conjugates whose DNA oligos are not complementary to the immobilized DNA fragments on the designated surface. Therefore, the concentration of the desired complexes, which have DNA oligos complimentary to the immobilized DNA fragments, is improved.

In certain embodiments, the solutions used for washing include any common biological buffer with a pH value range from 5 to 11, such as distilled water, PBS solution, etc. In certain embodiments, the solutions used for washing contain a higher concentration of salt and less than 2% of mild detergents such as Tween20, Triton X-100, etc.

During stage (D), the 3′ end of the first DNA oligo and the 5′ end of the second DNA oligo are ligated to become one oligo. Because the first and second antibodies have bound to the target protein, and the 5′ end of the first DNA oligo and the 3′ end of the second DNA oligo are close enough to be ligated. After the ligation of the two DNA oligos, the target protein, the first and second antibodies, the first and second DNA oligos form a closed loop structure.

After the ligation is accomplished on the designated surface, the complexes formed are removed from the designated surface and the DNA fragments are release by increasing the temperature to about 95C (antibody-DNA complex is denatured at temperature higher than 70C). The antigen-antibody-DNA complexes bound to the immobilized DNA fragments are removed from the designated surface when the temperature is increase to 95C. Since the ligation is done, one can heat up the plate to denature the complex and to release the ligation products (full length DNA) into the solution for qPCR reaction.

During the stage (D), an ELISA-PCR test is conducted for the antigen-antibody-DNA complexes removed from the designated surface. In certain embodiments, a primer pair which are specific for the two DNA oligos are used. One primer of the primer pair is complementary and binds to the sequence having about 1-50 based pair at the 3′ end of the first DNA oligo. In one embodiment, the range for the base pair is about 10-40 bp. In one embodiment, the range for the base pair is about 15-35 bp. In one embodiment, the range for the base pair is about 20-30 bp. In one embodiment, the range for the base pair is about 18-25 bp. Following synthesis and at the end of the first cycle, each double-stranded DNA molecule consists of one new and one old DNA strand. The other primer of the primer pair is identical to the first 5-40 bases of the 5′ end of the second DNA oligo and will bind to the 3′ end of the newly synthesized DNA after the first cycle. PCR then continues with additional cycles that repeat the aforementioned steps. The newly synthesized DNA segments serve as templates in later cycles, which allow the DNA target to be exponentially amplified millions of times.

A regular real time PCR test is then performed. If the first and second antibodies both bind to the target protein, and the 3′ end of the first DNA oligo and the 5′ end of the second DNA oligo are ligated, the PCR test would produce amplification DNA sequences, starting with the primers.

Because all DNA polymerases possess 5′→3′ polymerase activity, which is the incorporation of nucleotides to extend primers at their 3′ ends in the 5′ to 3′ direction, the second DNA oligo whose 3′ end is attached to the second antibody should be amplified first and most. The amount of the first synthesis DNA molecules is correlated with the presence of the target protein. The more of the target protein presents, the quicker the PCR reaction will reach the detectable threshold—the Ct or Cq value of real-time PCR.

In one embodiment of the invention, both primers have 5-40 bases. One having ordinary skill in the art can design primers with different sequences according to the first and second DNA oligo sequences. That is, approximately half of the primers should be complementary and binding to the sequence at the 3′ end of the second DNA oligo, while the other half of the primers should be identical to the 5′ end of the first DNA oligo and will bind to the 3′ end of the newly synthesis DNA molecules.

Amplification of the loop structure with the primers reflects the existence of the target protein.

In one embodiment, a typical ELISA-PCR test protocol is provided as below in Table 1.

TABLE 1
Protocol for ELISA-PCR test.

| | |
|---|---|
| Step 1: | Antigen Standard Curve Preparation |
| 1a | Reconstitute Protein standard with Assay Dilution Buffer |
| 1b | Prepare standard 1 (5000 pg/mL) from reconstituted protein standard |
| 1c | Make serial 1:5 dilution for standard 2-7 in Assay Dilution Buffer, standard 8 |
| | is blank. |
| Step 2: | Preparation of Pair for Verification |
| | Record antibody conjugate A and B clone numbers for the pairs to be verified |
| | in Table 4 in Summary Report tab |
| 2a | Prepare each antibody pair by diluting Antibody Conjugate A and Antibody |
| | Conjugate B in Antibody Conjugate Dilution Buffer (1 to 60) |
| 2b | Aliquot each diluted antibody pair from step 2a into 4 wells (42 uL in each) |
| Step 3: | PLA Binding Plate Preparation and qPCR |
| 3a | To qPCR plate on a cold block, use multi-channel pipet, add 2 ul of Antigen |
| | standard 1-12 to the bottom of the wells in all rows |
| | To the same qPCR plate, use multi-channel pipet, add 2 ul of Antibody |
| | Conjugate A and B Pair mix to the wall of the wells in all columns. |
| | Seal plate and spin down. Incubate for 10 minutes to 1 hour at RT or overnight |
| | at 4 C. |
| 3b | Add 16 ul of PLA PCR master mix + Ligase to all reaction wells |
| | Run qPCR using Verification template. |
| Step 4: | Data Analysis |
| 4a | Once qPCR run is finished, export the raw data as .EDS file |
| 4b | Upload .EDS file to qPCR software for analysis |
| 4c | Standard curve plot, standard curve, and % bias will be generated. |

In one embodiment, antibodies specifically bind to a target protein of interest can be either lab-produced using techniques commonly known in the field, or obtained as commercial products.

### Blocking Nucleotide and Cleave Site

In certain embodiments, as shown in FIG. 1, a blocking nucleotide may be attached to an end of an immobilized DNA fragment which is not attached to the designated surface. The blocking nucleotide prevents the extension of the immobilized DNA fragment during qPCR reactions in the ELISA-PCR test.

In one embodiment, because the 5′ end of an immobilized DNA fragment is linked to the designated surface, the blocking nucleotide is attached to the 3′ end of the immobilized DNA fragment, as shown as “b” in FIG. 1.

In certain embodiments, the blocking nucleotide includes one of 3′-Spacer C3, 3′-Phosphat, 3′-dideoxy nucleotides (dideoxy cytidine), 3′-Inverted End and other nucleoside analogs. The nucleoside analogs are well-known class of DNA polymerase inhibitors. The nucleoside analogs resemble the natural nucleotides used by DNA polymerases but contain modifications that prevent further DNA elongation once incorporated into the DNA strand.

In certain embodiments, an immobilized DNA fragment may include a cleave site on the end which attaches to the designated surface. In one embodiment, the cleave site is at the 5′ end of the immobilized DNA fragment. The cleave site facilitates the release and potential re-capture of the complex using an alternative DNA fragment.

In one embodiment, the cleave site includes an uracil base to be cleave by UDG, or 8-oxoguanine to be cleaved by OGG1 or Fpg, or deoxyinosine to be cleaved by Endonuclease V, and etc.

### Additional Embodiments

In one embodiment, anti-antibody antibodies are used as an alternative capturing strategy in the Antibody-Antigen Capture ELISA-PCR test.

In one embodiment, the Antibody-Antigen Capture ELISA-PCR test of the present invention supports multiplexing detection of more than one antigen at the same time, by having more than one pair of DNA-antibody conjugates and more than one type of immobilized DNA fragment. Each type of immobilized DNA fragments specifically captures the corresponding pair of DNA-antibody conjugates and any complex formed by the DNA-antibody conjugates. For example, a first type of immobilized DNA fragments specifically binds to a first and/or second oligos of the DNA-antibody conjugates and thus only captures the DNA-antibody conjugates or any antigen-antibody-DNA complexes having the first and/or second oligos. Meanwhile, a second type of immobilized DNA fragments specifically binds to a third and/or fourth oligos of the DNA-antibody conjugates and thus only captures the DNA-antibody conjugates or any antigen-antibody-DNA complexes having the third and/or fourth oligos. By having such multi-specificity, the present invention is capable of simultaneous detection of more than one target proteins.

In one embodiment, the present invention allows simultaneous processing of multiple antibody pairs in separate reactions using different sets of beads or wells for parallel assay execution.

### Advantages of Antibody-Antigen Capture ELISA-PCR

Antibody-Antigen Capture ELISA-PCR test further improves the sensitivity and specificity of the ELISA-PCR test.

Previously, compared to the traditional ELISA test, the ELISA-PCR test has a number of advantages. First, the ELISA-PCR test has a higher sensitivity than the traditional ELISA test. The ELISA-PCR test can detect the target proteins with 10-100 times of the sensitivity as compared to traditional enzyme-linked reactions. The ELISA-PCR test also has a higher specificity as compared to the traditional ELISA test, because there are two antibodies to bind to the target protein at the same time to form a closed DNA loop, so as to render subsequent nucleic acid amplification possible.

The Antibody-Antigen Capture ELISA-PCR test improves the ELISA-PCR by capturing the complexes onto the same designated surface, on which the ligation of first and second DNA oligos are facilitated due to a high concentration of the complex.

The capturing technique of the present invention is adaptable to various downstream applications such as ImmunoPCR, Proximity Ligation PCR, and other immuno-assays that employ antibody-DNA conjugates as probes, significantly advancing molecular diagnostics.

### EXAMPLES

Example antibodies for the DNA-antibody conjugates are described in Table 2 below.

Table 2. Antibodies used in the conjugates of ELISA-PCR Test

| Pair # | Antigen | Vendor | Cat Number | Clone | Source |
|---|---|---|---|---|---|
| | Tau | R&D Systems | MAB106291 | 1032501 | mouse IgG |
| | | R&D Systems | MAB3494 | 376720 | mouse IgG |
| | Tau | Thermo Fisher | MN1050 | AT270 | mouse IgG |
| | Thr181 | R&D Systems | MAB3494 | 376720 | mouse IgG |
| | cTNI | Creative | CABT-L1129 | na | mouse IgG |
| | | Diagnostics | | | |
| | | Creative | CABT-L1130 | na | mouse IgG |
| | | Diagnostics | | | |
| | cTNT | Creative | CABT-L702 | TD75-14 | Rabbit IgG |
| | | Diagnostics | | | |
| | | Creative | DMAB1823MH | 2G3 | mouse IgG |
| | | Diagnostics | | | |

Other examples may include:
Tau monoclonal antibody MAB3494 (Clone 376720)
Tau Thr181 specific antibody MN1050, (Clone AT270),
Tau monoclonal antibody MAB106291 (Clone 1032501)
Examples of DNA oligos and primers are listed as below.

Table 3. Examples of DNA oligos the DNA-antibody conjugates, their complementary immobilized DNA fragments, and their corresponding primers

| | | | | Capture | |
|---|---|---|---|---|---|
| | | Forward | Reverse | DNA | |
| Oligo A | Oligo B | Primer | Primer | molecule | |
| Example | aattggacgt | ccgcgcatcc | aattggacgt | cgtattccat | cgtattccat |
| 1 | ggctttcgcg | tatgtatcaa | ggctttcgcg | tagaactaac | tagaactaac |
| | ttagtacgta | gttagttcta | ttagt | ttgat | -Biotin |
| | gcatggtcac | atggaatacg | (SEQ ID | (SEQ ID | (SEQ ID |
| | acaagcacag | (SEQ ID | No. 41) | No. 61) | No. 81) |
| | tagatcctgc | No. 21) | | | |
| | (SEQ ID | | | | |
| | No. 1) | | | | |
| Example | tcataaggag | ggcggtaagg | tcataaggag | tgtcttaccg | tgtcttaccg |
| 2 | tccggtgtag | tatcactcaa | tccggtgtag | agcctgcttc | agcctgcttc |
| | cgaaagatca | gaagcaggct | cgaaa | ttgag | -Biotin |
| | aggcgaccct | cggtaagaca | (SEQ ID | (SEQ ID | (SEQ ID |
| | aggtagcaac | (SEQ ID | No. 42) | No. 62) | No. 82) |
| | cgccggctcc | No. 22) | | | |
| | (SEQ ID | | | | |
| | No. 2) | | | | |
| Example | cttccgctgg | gtgactatct | cttccgctgg | ggatcggcag | ggatcggcag |
| 3 | gatccaacgt | gtgccagatc | gatccaacgt | tctgccagac | tctgccagac |
| | tggcggccga | gtctggcaga | tggcg | gatct | -Biotin |
| | agccgccatt | ctgccgatcc | (SEQ ID | (SEQ ID | (SEQ ID |
| | ccatagtgag | (SEQ ID | No. 43) | No. 63) | No. 83) |
| | tccttcgtct | No. 23) | | | |
| | (SEQ ID | | | | |
| | No. 3) | | | | |
| Example | tcctagatac | cctgacctaa | tcctagatac | cggcggagcc | cggcggagcc |
| 4 | cgcactctgg | cggtaagagg | cgcactctgg | attatgtgag | attatgtgag |
| | gcagtacgag | ctcacataat | gcagt | cctct | -Biotin |
| | gtaatgccag | ggctccgccg | (SEQ ID | (SEQ ID | (SEQ ID |
| | tcacccagtg | (SEQ ID | No. 44) | No. 64) | No. 84) |
| | ccgaacaaca | No. 24) | | | |
| | (SEQ ID | | | | |
| | No. 4) | | | | |
| Example | tccagttcgg | tagcgtgacg | tccagttcgg | tacgatcgca | tacgatcgca |
| 5 | tcagtgggtc | gccgcagggg | tcagtgggtc | ttttatgggt | ttttatgggt |
| | actgcaagta | acccataaaa | actgc | cccct | -Biotin |
| | gtcgattgca | tgcgatcgta | (SEQ ID | (SEQ ID | (SEQ ID |
| | ttgccaatct | (SEQ ID | No. 45) | No. 65) | No. 85) |
| | ccgagtgatt | No. 25) | | | |
| | (SEQ ID | | | | |
| | No. 5) | | | | |
| Example | aaaatcacca | gtaatgaata | aaaatcacca | tcgtccctct | tcgtccctct |
| 6 | gtgcccaaga | ttcagtagaa | gtgcccaaga | aacacagact | aacacagac- |
| | ccaggggggc | agtctgtgtt | ccagg | ttcta | Biotin |
| | tcgccgcgtt | agagggacga | (SEQ ID | (SEQ ID | (SEQ ID |
| | ggctaatccc | (SEQ ID | No. 46) | No. 66) | No. 86) |
| | ggtacatctt | No. 26) | | | |
| | (SEQ ID | | | | |
| | No. 6) | | | | |
| Example | cgagcggcgc | tatcttcctg | cgagcggcgc | tagcaacagc | tagcaacagc |
| 7 | agccgattag | cccagtggcg | agccgattag | gataaccatc | gataaccatc |
| | gaccatgtag | gatggttatc | gacca | cgcca | -Biotin |
| | aacatttgtt | gctgttgcta | (SEQ ID | (SEQ ID | (SEQ ID |
| | acaagacttc | (SEQ ID | No. 47) | No. 67) | No. 87) |
| | ttttaaacac | No. 27) | | | |
| | (SEQ ID | | | | |
| | No. 7) | | | | |
| Example | gcttagttca | gtgcggacgg | gcttagttca | cggtcagatt | cggtcagatt |
| 8 | acctcgaata | cgttgcaact | acctcgaata | aggccctgga | aggccctgga |
| | cctcgtatca | tccagggcct | cctcg | agttg | -Biotin |
| | ttgtgcacct | aatctgaccg | (SEQ ID | (SEQ ID | (SEQ ID |
| | gccggtcacc | (SEQ ID | No. 48) | No. 68) | No. 88) |
| | agccaacgat | No. 28) | | | |
| | (SEQ ID | | | | |
| | No. 8) | | | | |
| Example | atgacatgtg | gactagagtg | atgacatgtg | gcataacggg | gcataacggg |
| 9 | gatgggcagt | gcgagaacta | gatgggcagt | aggccctggt | aggccctgg- |
| | ggccggttgt | tggcgtgtga | ggccg | agtt | Biotin |
| | tacacgcctg | cccgttatgc | (SEQ ID | (SEQ ID | (SEQ ID |
| | ccgcgacgct | (SEQ ID | No. 49) | No. 69) | No. 89) |
| | gaatgacccg | No. 29) | | | |
| | (SEQ ID | | | | |
| | No. 9) | | | | |
| Example | cggtctagct | gccttaggat | cggtctagct | atctcgacca | atctcgacca |
| 10 | gactgtctat | tcacttcagc | gactgtctat | ggccctggcg | ggccctggcg |
| | cgcctaggtc | gcgcaggcct | cgcct | ctga | ctga |
| | aaatagggag | gggtcgagat | (SEQ ID | (SEQ ID | (SEQ ID |
| | ctttgatatc | (SEQ ID | No. 50) | No. 70) | No. 90) |
| | tgcgtgtcca | No. 30) | | | |
| | (SEQ ID | | | | |
| | No. 10) | | | | |
| Example | agtttatccc | ggctcagctc | agtttatccc | ggcggaccag | ggcggaccag |
| 11 | accaaactat | tatttttgtg | accaaactat | aggccctggc | aggccctgg- |
| | agccgtacag | gtcatgggtt | agccg | acaa | Biotin |
| | gccgaaatct | ctggtccgcc | (SEQ ID | (SEQ ID | (SEQ ID |
| | taagtcatat | (SEQ ID | No. 51) | No. 71) | No. 91) |
| | cgcgcgacta | No. 31) | | | |
| | (SEQ ID | | | | |
| | No. 11) | | | | |
| Example | gcgtgcccag | cggaaacggg | gcgtgcccag | attttcgttt | attttcgttt |
| 12 | ggtatattag | tgcgtggact | ggtatattag | aggccctgga | aggccctgg- |
| | gtcagcatcg | agcgaggagc | gtcag | gtcc | Biotin |
| | gatggactga | aaacgaaaat | (SEQ ID | (SEQ ID | (SEQ ID |
| | catgaacctt | (SEQ ID | No. 52) | No. 72) | No. 92) |
| | tacaccgaag | No. 32) | | | |
| | (SEQ ID | | | | |
| | No. 12) | | | | |
| Example | gtccacccga | cgtttgcatt | gtccacccga | ccttatgctc | ccttatgctc |
| 13 | ccgtacatag | taagggccgc | ccgtacatag | aggccctggg | aggccctgg- |
| | aaatgagggt | acgaaccaca | aaatg | cggc | Biotin |
| | ccccgtacgc | gagcataagg | (SEQ ID | (SEQ ID | (SEQ ID |
| | ccacgcacct | (SEQ ID | No. 53) | No. 73) | No. 93) |
| | gttcgctcgt | No. 33) | | | |
| | (SEQ ID | | | | |
| | No. 13) | | | | |
| Example | gtcaccatgt | ggtttacgtg | gtcaccatgt | cctagcctgt | cctagcctgt |
| 14 | accaagggcg | ggaaaggtgc | accaagggcg | aggccctggg | aggccctgg- |
| | ataacgatcg | ttgtgtccca | ataac | cacc | Biotin |
| | gtgggagtat | acaggctagg | (SEQ ID | (SEQ ID | (SEQ ID |
| | tcatcgtggt | (SEQ ID | No. 54) | No. 74) | No. 94) |
| | gaagacgctg | No. 34) | | | |
| | (SEQ ID | | | | |
| | No. 14) | | | | |
| Example | gccggcgtgg | gctgtccgat | gccggcgtgg | gccaacccca | gccaacccca |
| 15 | aaggtaacag | cgtatattag | aaggtaacag | aggccctggc | aggccctgg- |
| | caccgctgcg | gactccgcga | caccg | ctaat | Biotin |
| | agcctaatgc | tggggttggc | (SEQ ID | (SEQ ID | (SEQ ID |
| | gccgtttcca | (SEQ ID | No. 55) | No. 75) | No. 95) |
| | cgaacacagg | No. 35) | | | |
| | (SEQ ID | | | | |
| | No. 15) | | | | |
| Example | tcctggcctg | ggaagaggtc | tcctggcctg | ggagagtagc | ggagagtagc |
| 16 | cttgatgtct | gccctacaaa | cttgatgtct | aggccctggt | aggccctg- |
| | cgtgaccttc | atagatttgc | cgtga | ttgt | Biotin |
| | ttagagatgg | gctactctcc | (SEQ ID | (SEQ ID | (SEQ ID |
| | acgaaatgtt | (SEQ ID | No. 56) | No. 76) | No. 96) |
| | tcgcgaccta | No. 36) | | | |
| | (SEQ ID | | | | |
| | No. 16) | | | | |
| Example | aggacctcta | cgcaatgacg | aggacctcta | caggaaccga | caggaaccga |
| 17 | gctcctttac | gacgtattcc | gctcctttac | ggccctggag | ggccctgga- |
| | aaagtgctgg | tctggccaca | aaagt | gaat | Biotin |
| | ttccctttcc | tcggttcctg | (SEQ ID | (SEQ ID | (SEQ ID |
| | ggcgggatgc | (SEQ ID | No. 57) | No. 77) | No. 97) |
| | cttatctaaa | No. 37) | | | |
| | (SEQ ID | | | | |
| | No. 17) | | | | |
| Example | gtataatgct | caacgtgtcc | gtataatgct | ctcggccttg | ctcggccttg |
| 18 | ggagccctcc | gtgttcacgt | ggagccctcc | aggccctgga | aggccctgg- |
| | cccaagcgtt | tatatgcgca | cccaa | cgtg | Biotin |
| | cagggtgggg | caaggccgag | (SEQ ID | (SEQ ID | (SEQ ID |
| | tttgctacga | (SEQ ID | No. 58) | No. 78) | No. 98) |
| | cttccgagtc | No. 38) | | | |
| | (SEQ ID | | | | |
| | No. 18) | | | | |
| Example | aagtggcagc | gtgttccggc | aagtggcagc | gcatgattga | gcatgattga |
| 19 | ctaaacgatg | tgttatcctg | ctaaacgatg | aggccctggc | aggccctgg- |
| | ttgggggctc | catcggaacg | ttggg | agga | Biotin |
| | gcgatgcaca | tcaatcatgc | (SEQ ID | (SEQ ID | (SEQ ID |
| | cgctctggta | (SEQ ID | No. 59) | No. 79) | No. 99) |
| | caatacatac | No. 39) | | | |
| | (SEQ ID | | | | |
| | No. 19) | | | | |
| Example | caaaagcttt | ttccactgtg | caaaagcttt | acgctttgcg | acgctttgcg |
| 20 | aaacgcgagt | tttgtctcat | aaacgcgagt | aggccctgga | aggccctgg- |
| | tcccgcccat | gtaggacggg | tcccg | tgag | Biotin |
| | aacctggacc | cgcaaagcgt | (SEQ ID | (SEQ ID | (SEQ ID |
| | gaatgcggga | (SEQ ID | No. 60) | No. 80) | No. 100) |
| | tcatgcatcg | No. 40) | | | |
| | (SEQ ID | | | | |
| | No. 20) | | | | |

### Example 1: Surface-Capture ELISA-PCR Test of ST2 (ST2)

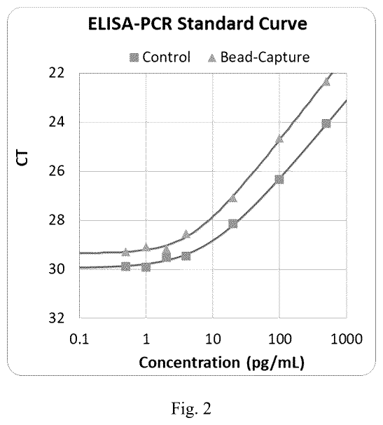

FIG. 2 and Table 5 show the results of detection of ST2 using the Surface-Capture (antibody-antigen capture) Elisa-PCR tests.

In particular, as shown, the Surface-Capture Elisa-PCR test for ST2ST2 as the target antibody is conducted according to the procedure described as following:

1. Prepare the antigen standard on ice according to Table 4 below:

TABLE 4
Antigen standards

| Concentrations | Standards | Calculation |
|---|---|---|
| 500 | STD1- | 199 ul ADB + 1 ul of 1 ug/ml ST2 (1 uL Stock + 9 uL ADB) |
| 100 | STD2- | 20 ul of STD1 into 80 ul ADB |
| 20 | STD3- | 20 ul of STD2 into 80 ul ADB |
| 4 | STD4- | 40 ul of STD3 into 160 ul ADB |
| 2 | STD5- | 80 ul of STD4 into 80 ul ADB |
| 1 | STD6- | 80 ul of STD5 into 80 ul ADB |
| 0.5 | STD7- | 80 ul of STD6 into 80 ul ADB |
| 0.0000 | BKG | 80 ul ADB |

### 2. Prepare ST2 Probe Mix on ice:

add 10 μL of 3′-ST2 probe stock and 10 ul 5′-ST2 probe stock to 500 ul ACDB.

### 3. Prepare the Binding mix:

Mixed 50 ul of STD with 50 ul of probe mix. Incubate for 2 hour at room temperature
   4. Incubate Biding mix with Beads C1 for antibody-antigent complex capturing:
100 uL binding mix was incubated with 10 ul of Beads C1 (containing the capturing DNA oligo). Incubate for 1 hour at room temperature with shaking at 700 rpm.
   5. Beads washes and ELISA-PCR reaction:
The binding mix+beads retained at magnetic rack was washed for 2× with (20% ACDB/20% ADB, 150 μL, 10× finger flick/2 min stand) to remove the excess of non-binding magnetic biotinylated beads.
After 2 washes, the retained beads on the magnetic rack were resuspended in 40 ul 20% ACDB/20% ADB.
10 ul of solution in triplicated was placed with 10 ul MM2x.
Perform ELISA-PCR in qPCR instrument as list earlier.
   The target protein, ST2, is made into a collection of samples having a gradient concentration: 500 pg/ml, 100 pg/ml, 20 pg/ml, 4 pg/ml, 2 pg/ml, 1 pg/ml, 0.5 pg/ml, and 0 pg/ml.

A control group is conducted using ELISA-PCR test without the surface capture technique. That is, only stage (D) is performed using a mixture of target proteins and the DNA-oligo conjugates, without performing the stages (A)-(C) using a designated surface having immobilized DNA fragments.

As it can be seen in the FIG. 2 and Table 5, while both the control group and surface capturing group demonstrate a curve corresponding to the gradient concentration of the ST2 in the collection of samples, the surface capturing group demonstrates a lower Ct value as compared to the control group. This lower Ct value shows that the surface capturing group has an increased sensitivity for detection and an improved limit of detection (LOD). In particular, as shown in Table 5, the surface capturing group demonstrates a LOD of 0.97 pg/ml, while the control group demonstrates a LOD of 2.90 pg/ml.

TABLE 5
Detection of ST2 using the Surface-Capture Elisa-PCR tests

| | |
|---|---|
| ST2 Conc | CT |

| | (pg/mL) | Control | Surface-Capture |
|---|---|---|---|
| Standard 1 | 500 | 24.08 | 22.34 |
| Standard 2 | 100 | 26.35 | 24.67 |
| Standard 3 | 20 | 28.17 | 27.10 |
| Standard 4 | 4 | 29.47 | 28.56 |
| Standard 5 | 2 | 29.54 | 29.19 |
| Standard 6 | 1 | 29.92 | 29.10 |
| Standard 7 | 0.5 | 29.90 | 29.31 |
| NTC | 0 | 30.02 | 29.37 |
| | LOD | 2.90 | 0.97 |
| | (pg/mL) | | |

### Example 2: Surface-Capture ELISA-PCR Test of Tau181

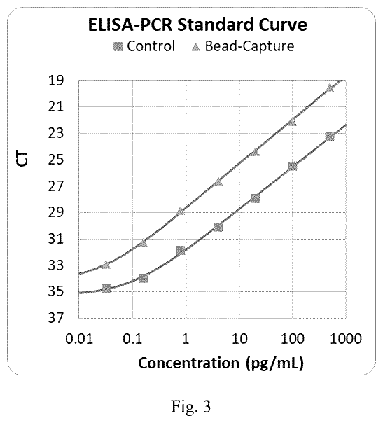

FIG. 3 and Table 6 show the results of detection of Tau181 using the Surface-Capture (antibody-antigen capture) Elisa-PCR tests.

In particular, as shown, the Surface-Capture Elisa-PCR test for Tau181 as the target antibody is conducted according to the procedure described above.

The target protein, Tau181, is made into a collection of samples having a gradient concentration: 500 pg/ml, 100 pg/ml, 20 pg/ml, 4 pg/ml, 2 pg/ml, 1 pg/ml, 0.5 pg/ml, and 0 pg/ml.

A control group is conducted using ELISA-PCR test without the surface capture technique. That is, only stage (D) is performed using a mixture of target proteins and the DNA-oligo conjugates, without performing the stages (A)-(C) using a designated surface having immobilized DNA fragments.

As it can be seen in the FIG. 3 and Table 6, while both the control group and surface capturing group demonstrate a curve corresponding to the gradient concentration of the Tau181 in the collection of samples, the surface capturing group demonstrates a lower Ct value as compared to the control group. This lower Ct value shows that the surface capturing group has an increased sensitivity for detection and an improved limit of detection (LOD). In particular, as shown in Table 6, the surface capturing group demonstrates a LOD of 0.02 pg/ml, while the control group demonstrates a LOD of 0.004 pg/ml.

TABLE 6
Detection of Tau181 using the Surface-Capture Elisa-PCR tests

| | |
|---|---|
| Tau181 Conc | CT |

| | (pg/mL) | Control | Surface-Capture |
|---|---|---|---|
| Standard 1 | 500 | 23.29 | 19.49 |
| Standard 2 | 100 | 25.49 | 22.10 |
| Standard 3 | 20 | 27.93 | 24.41 |
| Standard 4 | 4 | 30.09 | 26.62 |
| Standard 5 | 0.8 | 31.85 | 28.86 |
| Standard 6 | 0.16 | 33.99 | 31.27 |
| Standard 7 | 0.032 | 34.77 | 32.92 |
| NTC | 0 | 35.30 | 33.99 |
| | LOD (pg/mL) | 0.02 | 0.004 |

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments were chosen and described to explain the principles of the invention and their practical application so as to enable others skilled in the art to utilize the invention and various embodiments and with various modifications as are suited to the particular use contemplated. Alternative embodiments will become apparent to those skilled in the art to which the present invention pertains without departing from its spirit and scope. Accordingly, the scope of the present invention is defined by the appended claims rather than the foregoing description and the exemplary embodiments described therein.

Some references, which may include patents, patent applications and various publications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entireties and to the same extent as if each reference was individually incorporated by reference.

What is claimed is:

1. An antibody-antigen capture ELISA-PCR test set for detection of a target protein, comprising:
a first antibody-DNA conjugate having a first antibody which is configured to bind to a target protein; and a first DNA oligo linked to the first antibody;
a second antibody-DNA conjugate having a second antibody which is configured to bind to the target protein; and a second DNA oligo linked to the second antibody; and
multiple immobilized DNA fragments attached to a designated surface on their attaching ends,
wherein the immobilized DNA fragments are complementary to one of the first DNA oligo and the second DNA oligo.

2. The antibody-antigen capture ELISA-PCR test set according to claim 1, wherein the first antibody binds to a first binding site of the target protein and the second antibody binds to a second binding site of the target protein.

3. The antibody-antigen capture ELISA-PCR test set according to claim 1, wherein the attaching ends of the immobilized DNA fragments are their 5′ ends.

4. The antibody-antigen capture ELISA-PCR test set according to claim 1, wherein the immobilized DNA fragments are configured to capture a complex formed by the first antibody-DNA conjugate, the second antibody-DNA conjugate, and the target protein.

5. The antibody-antigen capture ELISA-PCR test set according to claim 1, wherein a blocking nucleotide is attached to each of the immobilized DNA fragments on an end opposite to its attaching end.

6. The antibody-antigen capture ELISA-PCR test set according to claim 1, wherein each of the immobilized DNA fragments comprises a cleave site adjacent to the attaching end.

7. The antibody-antigen capture ELISA-PCR test set according to claim 1, wherein each of the immobilized DNA fragments has a length between 15-30 nucleotide bases.

8. The antibody-antigen capture ELISA-PCR test set according to claim 1, wherein each of the immobilized DNA fragments is attached to the designated surface via click chemistry interaction or avidin-biotin interactions.

9. The antibody-antigen capture ELISA-PCR test set according to claim 1, wherein the first DNA oligo is covalently linked to the first antibody, and the second DNA oligo is covalently linked to the second antibody.

10. The antibody-antigen capture ELISA-PCR test set according to claim 1, wherein the first DNA oligo is covalently linked to the first antibody on its 3′ end, and the second DNA oligo is covalently linked to the second antibody on its 5′ end.

11. The antibody-antigen capture ELISA-PCR test set according to claim 1, wherein the first DNA oligo and second DNA oligo each has a length between 20-300 nucleotide bases.

12. The antibody-antigen capture ELISA-PCR test set according to claim 1, wherein when the first antibody and second antibody each binds to the target protein, the 5′ end of the first DNA oligo and the 3′ end of the second DNA oligo attaches to each other.

13. The antibody-antigen capture ELISA-PCR test set according to claim 12, wherein the first antibody and second antibody, the first DNA oligo and second DNA oligo, and the target protein form a close loop structure.

14. The antibody-antigen capture ELISA-PCR test set according to claim 1, wherein the target protein is selected from the group consisting of marker proteins of pathogens, marker proteins of infectious diseases; marker proteins of genetic diseases, marker proteins of acquired diseases.

15. A method for quantitatively detecting a target protein using antibody-antigen capture ELISA-PCR test, comprising:
mixing a first antibody-DNA conjugate, a second antibody-DNA conjugate, and a sample having the target protein, to form a complex mixture;
incubating the complex mixture on a designated surface on which immobilized DNA fragments are attached;
washing the incubated designated surface and collected a concentrated complex mixture; and
conduct an ELISA-PCR test using the concentrated complex mixture.

16. The method according to claim 15, wherein
the first antibody-DNA conjugate having a first antibody which is configured to bind to a first biding site of the target protein and a first DNA oligo linked to the first antibody;
the second antibody-DNA conjugate having a second antibody which is configured to bind to a second biding site of the target protein; and a second DNA oligo linked to the second antibody.

17. The method according to claim 16, wherein when the first antibody and second antibody each binds to the target protein, a 3′ end of the first DNA oligo and a 5′ end of the second DNA oligo attach to each other.

18. The method according to claim 17, wherein the ELISA-PCR test comprises
adding a first primer and a second primer to the sample, wherein the first primer is complementary to a sequence at the 3′ end of the first DNA oligo, and the second primer is identical to a sequence at the 5′ end of the second DNA oligo.

19. The method according to claim 18, wherein the ELISA-PCR test further comprises
starting PCR cycle to amplify the first and second DNA oligos with the first and second primers.

20. The method according to claim 19, wherein the ELISA-PCR test further comprises
quantitatively analyzing the result of the PCR amplification to determine the amount of the target protein in the sample.
